(19) United States
(12) Patent Application Publication
(10) Pub. No.: US 2001/0007153 A1
(43) Pub. Date: Jul. 5, 2001

(54) SOLID CHIMERIC ORGANS, ANIMAL MODELS HAVING SAME, PROCESS FOR PREPARING SAME, NON-TUMORIGENIC IMMORTALIZED HUMAN CELL LINES, SUSCEPTIBLE CELLS AND CYTOPATHIC MAMMALIAN VIRUSES

(76) Inventors: JENNIFER JUNE BROWN, BRONX, NY (US); ELAZAR RABBANI, NEW YORK, NY (US); JAMES J. DONEGAN, LONG BEACH, NY (US); JAYANTA ROY-CHOWDHURY, NEW ROCHELLE, NY (US)

Correspondence Address: ENZO THERAPEUTICS INC, ENZO BIOCHEM INC, 9TH FLOOR, 527 MADISON AVENUE, NEW YORK, NY, 10022

(21) Appl. No.: 08/876,635

(22) Filed: Jun. 16, 1997

(51) Int. Cl. A01K 67/00; A01K 67/033; A01K 67/027

(52) U.S. Cl. 800/8; 800/9; 800/10; 800/11; 800/12; 800/13; 800/14; 800/18

(57) ABSTRACT

This invention provides useful solid chimeric organs as well as animal models having such solid chimeric organs and processes for their preparation. Such organs find significant value and use in developing new models for disease, drug and therapeutic investigations and monitoring, and in studying storage functions and processes. The solid chimeric organs are comprised of recipient cells and donor cells, the latter themselves comprising allogeneic or xenogeneic cells which are unmodified or modified to contain one or more nucleic acid segments capable of exhibiting at least one biological property, e.g. DNA synthesis, replication, promoter function, transcription, translation, reverse transcription, and the like, non-native to the donor cell. The solid chimeric organs can be prepared from recipient organs using recipient cells and implanted allogeneic or xenogeneic donor cells from non-homologous organs or tissues. Also provided by this invention are non-tumorigenic immortalized human cell lines modified by having sequences derived from hepatotrophic viruses introduced into the cells. Susceptible cells, cytopathic mammalian viruses, target cells rendered susceptible to cytopathic events, and human cells infected or transfected by noncytopathic viruses are also provided.

### FIELD OF THE INVENTION

This invention relates to the field of organ development and animal models for disease based upon new organs. This invention also relates to non-tumorigenic immortalized human cell lines and the derivation of susceptible cells from non-susceptible ones. Induction of cytopathic events by modifications to viruses, their hosts or their environment are also encompassed by the field of this invention.

All patents, patent applications, patent publications, scientific articles, and the like, cited or identified in this application are hereby incorporated by reference in their entirety in order to describe more fully the state of the art to which the present invention pertains.

### BACKGROUND OF THE INVENTION

The absence of an effective drug to treat human hepatitis B viral infection (HBV), which affects an estimated 400 million people worldwide, is due in part to the absence of model for HBV that duplicates all the conditions of infection in humans. Existing animal models of HBV infection are limited by the species-specificity of HBV and HBV-like animal viruses (hepadnaviruses) for infection. In nature HBV infects only humans, but experimentally, chimpanzees (Barker et al., J. Infect. Dis. 127:648-662 (1973)); and tree shrews, a lower order primate (Walter et al., Hepatology 24:1-5 (1996); Yan et al., J. Cancer Res Clin Oncol 122:283-288 (1996); and Yan et al., J. Cancer Res Clin Oncol 122:289-295 (1996)) can be infected. All of the foregoing publications are incorporated by reference. Chimpanzees are closely related to humans but are expensive to maintain and have relatively long reproductive cycles that hinder genetic manipulations. The use of tree shrews is limited by a lack of strains with appropriate genetic markers. In addition, although HBV is able to replicate in both chimpanzees and tree shrews, the level of infection, replication and expression is lower than in humans (Walter et al. (1996), supra), indicating that the infection of the animal hepatocytes is not identical to infection of human hepatocytes. Furthermore, the use of primates as test models involves high expenses in purchase and maintenance and as well as stringent regulations concerning their care and use.

In vivo studies of viruses related to HBV have been used as models for some of the processes involved in HBV infection. Two examples are duck hepatitits B virus (DHBV) infections in Pekin ducks (Long et al., Developments in Biological Standards 81:163-168 (1993); and Offensperger et al., Clinical Investigator 72:737-741 (1994)) and woodchuck hepatitis virus (WHV) infections in woodchucks (Rogler and Chisari, Seminars in Liver Disease, 12:265-278 (1992), Bannasch et al., Cancer Research 55:3318-3330 (1995)). All of the foregoing are incorporated by reference herein. However, DHBV and WHV differ significantly from HBV in their nucleic acid sequences; HBV DNA has only 70% homology with WHV and 40% homology with DHBV (Mandart et al., J. Virol. 49:782-792 (1984), Seeger et al., Science 232:477-484 (1986)), the contents of which are incorporated in this application. In addition, there are differences in the organization of equivalent open reading frames (ORFs) coding for viral proteins. These hepadnaviruses are also species specific and are incapable of crossinfecting humans or primates. Therefore, information gained from non-HBV hepadnaviruses model systems is limited in its application to all of the processes involved in HBV infection of humans.

To provide model systems of HBV replication, human cell lines have been transfected with HBV DNA. One transformed cell line, Hep G 2.2.15, has been described which has most of the features of a productive HBV infection with continuous synthesis of HBsAg and HBeAg as well as extracellular viral particles containing HBV DNA (Sells et al., Proc. Natl. Acad. Sci. (USA) 84:1005-1009 (1987)), incorporated by reference herein. Although this system has been used to test the efficacy of potential HBV therapeutic drugs (Furman et al., Antimicrobial Agents & Chemotherapy 36:2686-2692 (1992); and Nair et al., Antimicrobial Agents & Chemotherapy 39:1993-1999 (1995)), (incorporated by reference herein), this system as well as other HBV transfected cell lines have limitations in their predictive value in that they are in vitro systems and thereby lack features intrinsic to an in vivo HBV infection such as interactions with hepatic structure, the presence of hepatotrophic factors and immunoreactivity. This cell line as well as all other HBV containing cell lines also have the limitation that they are derived from liver tumor cells and therefore differ from the normal primary hepatocytes which are the hosts for HBV replication in vivo. Altered properties typical of tumor cells include abnormal growth characteristics, chromosomal rearrangements and aneuploid chromosome representation as well as differences in transportation and metabolism of potential therapeutic agents. Also, during HBV infection in humans, there is continuous transfer of progeny viruses to uninfected hepatocytes that perpetuates further rounds of replication. This process is lacking in cell lines with resident viral sequences since each cell has the HBV genome present and furthermore, the parental hepatoma cell line from which the transfected cell line was derived has previously been shown to be incapable of efficient infection by HBV viral particles.

To provide an in vivo model for HBV replication and expression, HBV DNA has been used to create transgenic mice whose liver cells continuously express HBV gene products and produce various replicative intermediates that are seen in a normal HBV infection. (Farza et al., J. Virol. 62:4144-4152 (1988), Araki et al., Proc. Natl. Acad. Sci. (USA) 86:207-211 (1989); DeLoia et al., J. Virol. 63:4069-4073 (1989); and Guidotti et al., J. Virol. 69:6158-6169 (1995)). This type of model has also been used to study the effect of drugs upon HBV replication and expression (Ueda et al., Virology 169:213-216 (1989); and Nagahata et al., Antimicrobial Agents and Chemotherapy 36:2042-2045 (1992)). All of the foregoing publications are incorporated by reference in this application. However, transgenic systems can not serve as complete models for HBV infection since they differ from the normal infective process in a number of ways. Transgenic systems are all based upon the presence of a genetically derived integrated copy of HBV in the chromosomal DNA. As such, they do not have substantial levels of covalently closed circular DNA (cccDNA) which is believed to be the major source of RNA transcription (Tuttleman et al., Cell 47:451-460 (1986), contents incorporated herein) as well as an important intermediary in normal HBV replication. This system also has the same limitations of a lack of an infective process descibed previously for the transfected cell lines because the human-specific viral particles released from the mouse hepatocytes are incapable of infecting mouse cells. In addition, there is no immune reaction to the HBV virus gene products in transgenic mice since they are not recognized as foreign. This is in contrast to a normal human infection where there is hepatic inflammation due to an immune reaction to HBV antigens in hepatocytes and this reaction is believed to be the causative agent for hepatocyte damage (Reviewed in Wright et al., Lancet 342:1340-1344 (1993), incorporated by reference herein). Lastly, mouse and human hepatocytes will not necessarily be equivalent in regards to either uptake or metabolism of potential therapeutic agents.

Viral growth in normal human hepatocytes in vivo would be a model that closer approximates a normal infection. To be an effective model this must be achieved by implantation of human cells into a host organism under conditions that emulate the natural surroundings of a hepatocyte. Most attempts to implant hepatocytes from one animal into another have been made in sites other than the liver due in part to a lack of morphological distinction between donor hepatocytes and host hepatocytes (Groth et al., Transplant Proc. 9:313-316 (1977), incorporated by reference herein). These ectopic sites have included skin, pancreas, peritoneum, beneath the renal capsule and in the splenic parenchyma (Reviewed in Gupta and Roy Chowdhury, Hepatology 15:156-162 (1992), also incorporated by reference). Although implanted ectopically as individual cells, hepatocytes can reorganize into structures that have some features resembling normal liver tissue (Kusano and Mito, Gastrenterology 82:616-628 (1982), incorporated by reference). To circumvent problems with implant survival and functionality, the implants have been supplemented with adjunctive elements such as pancreatic islets to provide localized trophic factors (Ricordi et al., Surgery 105:218-223 (1989)) and artificial matrices such as collagen coated microcarrier beads (Demetriou et al., Proc. Natl. Acad. Sci. (USA) 83; 7475-7479 (1986)). Both articles are incorporated by reference herein. However, these ectopic systems can never be considered to be functional equivalents of normal liver since they only approximate some of the structures found in liver, lack factors provided by portal blood and non-parenchymal cells in the liver and lack systemic interactions with related organs (spleen, gall bladder, bile ducts, etc.). This lack of equivalency is further evidenced by the the fact that the patterns and levels of mRNA expression are different for ectopically implanted hepatocytes as compared to hepatocytes implanted into the liver (Gupta et al., Human Gene Therapy 5:959-967 (1994), incorporated fully by reference herein).

To produce an in vivo model for investigating the effects of potential antiviral reagents on HBV infection, cells from the transfected cell line described previously, Hep G2.2.15, have been ectopically implanted into nude mice (Condreay et al., Antimicrobial Agents and Chemotherapy 38; 616-619 (1994), incorporated by reference into this application). In this model, both the limitations described for the HBV cell lines and the limitations derived from ectopic implantations apply.

The development of transgenic mice with markers expressed in hepatocytes has allowed the unequivocal identification of donor hepatocytes in implantations into the liver of congeneic recipients (Gupta et. al., Transplantation 50:472-475 (1990); and Ponder et al., Proc. Natl. Acad. Sci. (USA) 88:1217-1221 (1991)). Both are incorporated by reference into this disclosure. After intrasplenic injection of hepatocytes, as many as 1 donor cell was identified as engrafted in every 350 hepatocytes of the mouse liver (Ponder et al. (1991), supra). Transplantation of xenogeneic human cells into a mouse recipient liver containing normal hepatocytes has been performed in the context of a pre-clinical assessment that transplanted human hepatocytes would be viable and capable of engraftment in the recipient liver (Ledley et al., Pediatric Research 33:313-320 (1993), incorporated by reference). The implanted cells were identified by the presence of a stable non-toxic dye and the implanted cells represented about 1% of the liver mass. However, no appreciation was shown for any other utility besides a demonstration of the feasability of engraftment of human hepatocytes.

Liver-specific expression of a toxic gene in a transgenic mouse has been used to increase the efficiency of hepatocyte transplantation into the liver. Transgenic mice that contain a synthetic gene for urokinase-type plasminogen activator (uPA) undergo a replacement of transgene-expressing hepatocytes with clonally derived hepatocytes that have spontaneously deleted the transgene (Sandgren et al., Cell 66:245-256 (1991), also incorporated by reference). To test the feasability of transplanting xenogeneic liver cells into this transgenic system, uPA transgenic mice were bred with SCID mice to produce a SCID-uPA mouse strain. When normal rat hepatocytes were intrasplenically injected into the SCID-uPA mice, the mouse hepatocytes were replaced by the implanted rat cells in the liver (Rhim et al., Proc. Natl. Acad. Sci. USA 92:4942-4946 (1995), also incorporated by reference). Hybridization of rat and mouse specific probes for transferrin mRNA indicated that 90-100% of the native mouse hepatocytes were replaced by the non-native rat implants.

This animal model has the limitation that the transgene is hepatotoxic and although there is no large scale necrosis, the individual transgene hepatocytes show progressive degeneration (Sandgren et al. (1991), supra). The uPA transgenic newborn mice are unhealthy and suffer 50% mortality due to internal bleeding caused by the expression of the uPA transgene. Also, the replacement process does not in itself regenerate a normal liver; 100% of the uPA transgenic mice developed hepatocellular carcinomas due to DNA rearrangements that occurred as a consequence of the deletion of the hepatotoxic uPA gene (Sandgren et al., Proc. Natl. Acad. Sci. (USA) 89:11523-11527 (1992), incorporated by reference). As such, even when the native uPA hepatocytes have been mostly replaced by non-native donor cells, some residual host hepatocytes are present that could have undergone neoplastic transformation.

It was noted that when rat hepatocytes were used as the source of the donor cells in the uPA mouse system, the newly engrafted cells in the recipient's liver were morphologically indistinguishable from normal mouse hepatocytes (Rhim et al., (1995), supra) and there appeared to be appropriate replacement of detoxification and intermediary metabolism functions by the implanted non-native cells. Part of this overall functionality may be derived by the continuous existence of mouse derived support structures consisting of endothelial cells, biliary epithelial cells and fibroblasts that are capable of interacting appropriately with the implanted rat cells. However, when the donor is more phylogenetically distant from the recipient, the implanted cells may not be capable of appropriate interactions, and a complete replacement by non-native donor cells could create a non-functional liver which would be injurious to the health of the animal.

Other transplantation models of allogeneic or xenogeneic hepatocytes have involved gene replacement as therapy for genetic deficiencies. (For a review, see Gupta and RoyChowdhury, Hepatology 15:156-162 (1992), also incorporated into this application by reference) In these systems, the donor cells are unmodified cells that compensate for the genetic mutation present in the recipient cells. In these cases no non-native properties are being introduced into the recipient organ, but rather a function that is normally present in the cells of the organ of the recipient is being reconstituted by the implantation process.

Other problems hindering studies on HBV are derived from the nature of the HBV infection process. Although cytopathic effects are seen in the liver after HBV infection, this is believed to be an indirect effect mediated by the immune response to the infection. A lack of direct cytopathic effect by HBV is shown by the ability to create the cell lines and transgenic mice productive for HBV described earlier. After an infection in vitro, hepatocytes are viable and can be maintained and passaged while the culture is productive for replicative forms of HBV and viral particles (Lu et al., J. Vir. 70; 2277-2285 (1986), incorporated by reference herein). However, in studies of viral particle morphogenesis made by a related hepadnavirus, Duck Hepatitis B Virus (DHBV), the introduction of mutations in the p36 gene created variants that were directly cytopathic to the host cell when tested by in vitro transfections and/or infections (Lenhoff and Summers, J. Virol. 68:5706-5713 (1984), also incorporated by reference herein). An inverse relationship between the cytopathic effects of the variant and the ability to produce progeny virus was shown and the most cytopathic strains were unable to produce detectable levels of viral particles.

Other human diseases besides HBV lack suitable animal models due to restrictions in the host range for the pathogen. For instance, the exoerythrocytic stage of the life cycle of Plasmodium falciparum includes residence and propagation within hepatocytes. Due to species specific limitations of this pathogen, the causative agent for malaria, a model system has been devised that employed ectopic implantation of human hepatocytes underneath the kidney capsule in SCID mice (Sacci et al., Proc. Nat. Acad. Sci. (USA) 89; 3701-3705 (1992), incorporated fully herein by reference). Although there was visual identification of the presence of sporozoites and antigen staining for malarial proteins, this sytem also uses an ectopic location for the hepatocytes and therefore has the limitations intrinsic to such a location.

The present invention overcomes many of the problems in previously described surrogate models for HBV infection and therapy. In addition, the present invention is useful for other systems that require surrogate models. Modifications of both pathogens and host cells are described that are useful in testing therapeutic agents, isolation of cells resistant to viral infection or organ depletion.

### SUMMARY OF THE INVENTION

The present invention provides novel solid chimeric organs useful for developing new animal models for disease, for investigating and evaluating therapeutic regimens, and for storage functions and processes. The solid chimeric organ provided by the present invention comprises i) recipient cells; and ii) donor cells. The latter comprise allogeneic or xenogeneic cells that have been modified to contain one or more nucleic acid segments. Such segments are capable of exhibiting at least one biological property, e.g., DNA processes, such as synthesis, replication, transcription, translation, and the like. The biological property is non-native to said donor cell. Also provided is a non-human animal model having such a chimeric organ. This model is useful for studying viral and pathogenic infections, as well for investigating therapeutic means for combatting such infections.

The present invention also provides a solid chimeric organ prepared from a recipient organ. The solid chimeric organ comprises (i) recipient cells, and (ii) implanted allogeneic or xenogeneic donor cells from a non-homologous organ or tissue. In this form of the solid chimeric organ, the recipient organ has been reduced in size or the total number of recipient cells in the recipient organ have been reduced prior to or during implantation of the donor cells.

Also provided by this invention is a process for preparing a solid chimeric organ. The preparation process comprises the following steps. First, cells from an organ or tissue of a donor are removed. Into the removed donor cells are introduced one or more non-native nucleic acid segments or one or more native nucleic acid segments capable of non-native expression in a target cell. The non- native nucleic acid segments or the native nucleic acid segments are capable of eliciting at least one biological effect non-native to the donor cell. The donor cells are then implanted into a desired organ of an allogeneic or xenogeneic host. In a variation, the non-native nucleic acid segments or native nucleic acid segments capable of non-native expression can be introduced into the donor cells following the latters implantation into the desired organ.

Still yet provided by this invention is a process for preparing a solid chimeric organ from a recipient organ which comprises recipient cells. The following steps make up this process. The size of the recipient organ or the total number of recipient cells in the recipient organ is reduced. Next, allogeneic or xenogeneic donor cells from a non-homologous organ or tissue are provided. These allogeneic or xenogeneic donor cells are implanted into the recipient organ.

Another aspect of this invention concerns the non-tumorigenic immortalized human cell line which has been modified by introducing an hepatotrophic viral DNA sequence.

A yet further aspect provided by this invention is a susceptible cell derived from a non-susceptible cell that is a host in vivo for a virus or pathogen of interest. The non-susceptible cell has been modified to render it susceptible to infection by such virus or pathogen of interest. The modification is carried out by introducing into the cell one or more non-native nucleic acid segments or one or more native nucleic acid segments capable of non-native expression.

The invention herein provides an animal model having a susceptible cell derived from a non-susceptible cell that has been modified to render it susceptible to a viral or pathogenic infection, such modification being carried out inter alia by implantation, for example, orthotopic implantation.

The invention further provides a susceptible cell derived from a non-susceptible cell that has been modified to render it susceptible to a viral or pathogenic infection. Such a modification can be carried out by exposing the cell to any of the following elements: whole cells, cellular membranes, extracellular matrix molecules, and soluble extracts or purified components therefrom, or any combinations thereof.

The invention also provides a cytopathic mammalian virus derived from a non-cytopathic virus by any of the following: mutation, introducing additional promoters, incorporating other viral nucleic acid sequences or incorporating host cell nucleic acid sequences.

The invention further provides a target cell which has been rendered susceptible to a cytopathic event upon the infection by a noncytopathic virus. The target cell has been modified by introducing at least one native or non-native nucleic acid construct.

Also provided herein is a human cell infected or transfected by a noncytopathic virus. The human cell has been rendered susceptible to a cytopathic event by exposing it to any of the following biologically active compounds: cytokines, lymphokines, extracellular matrices, hormones, apoptosis factors, immunological factors, or a combination of any of the foregoing.

Another aspect of this invention is a process of increasing implantation efficiency. The process involves providing a recipient subject having an organ treated to reduce its mass or volume; and implanting allogeneic or xenogeneic donor cells into the treated organ. The donor cell has been modified to contain one or more non-native nucleic acid segments.

More particular details and embodiments of the invention are described fully below in the detailed description and preferred embodiments sections of this application that follow.

### BRIEF DESCRIPTION OF THE FIGURES

This disclosure contains no figures.

### DETAILED DESCRIPTION OF THE INVENTION

A method and composition is presented for an animal model that has a native solid organ transformed into a chimeric solid organ that comprises allogeneic or xenogeneic cells as well as native cells. The chimeric organ provides a system where the implanted cells are in an environment that provides endogenous stuctures and co-factors that could interact with the implanted cells. This is in contrast to previous methods that have used ectopic locations, where the donor cells are located in an environment and location that is physically distinct from the native cells of the target organ. Also, in contrast to previous methods that essentially involve the replacement of native cells of an organ by allogeneic or xenogeneic cells, the present invention allows the natural functions of the organ to be carried out by native cells while allowing additional properties to be expressed by the implanted allogeneic or xenogeneic components of the solid chimeric organ. The incorporation and propagation of the implanted cells in the chimeric organ creates an environment that provides the same conditions of the organ from whence the implanted allogeneic xenogeneic cells originated.

In a significant aspect, therefore, the present invention provides a solid chimeric organ comprising: i) recipient cells; and ii) donor cells comprising allogeneic or xenogeneic cells. The donor cells can themselves be chimeric if desired. In such a situation, the chimeric donor cells can be derived from a first and second parental cells which are phenotypically different from each other, and furthermore, the chimeric donor cells should exhibit at least two phenotypic characteristics. At least one of the phenotypic characteristics is derived from the first parental cell and at least one other phenotypic characteristic is derived from the second parental cell. The first or second parental cells are allogeneic or xenogeneic with respect to the recipient cells.

The allogeneic or xenogeneic cells of the chimeric organ can be unmodified cells or the cells could be usefully modified to contain at least one nucleic acid segment capable of exhibiting at least one biological property non-native to the donor cell. The list of biological properties useful in connection therewith and in the present invention is quite lengthy and will include any or all of the following or combinations of the following: DNA synthesis, DNA replication, promoter function, DNA transcription, DNA translation, reverse transcription, ligation, post-transcriptional modification, transcriptional processing, post-translational processing and protein export. The nucleic acid segments can be one or more non-native nucleic acid segments or one or more native nucleic acid segments capable of non-native expression in the donor cells. The nucleic acid segments used for preparing the solid chimeric organs are produced by or derived from any of the following or combinations of the following: one or more nucleic acid constructs, a viral vector, a mutated virus or wild type virus, modified or unmodified oligo- or polynucleotides and a chromosome or chromosomal segment. In the case where a chromosome or chromosomal segment is used, the chromosome or chromosomal segment may be taken from a cell taken from a different donor or taken from a cell from a different organ in the recipient.

Such native or non-native nucleic acid segment can be introduced into the allogeneic or xenogeneic cell either prior to or after its incorporation into a chimeric organ, or even during the formation of the chimeric organ. The non-native nucleic acid segment can be introduced into the allogeneic or xenogeneic cells by viruses, viral vectors, artificial constructs or any combination thereof. Viruses can include but are not limited to HBV, HCV, adenovirus, Adeno Associated Virus (AAV) and retroviruses such as MMLV. The non-native segments are nucleic acid polymers which can be single-stranded, double-stranded or partially double-stranded. They can be RNA or DNA, and they can be modified or unmodified as described in one or more of the following U.S. patents or U.S. patent applications: U.S. Pat. Nos. 4,711,955; 5,328,824; 5,449,767; 5,476,928; 5,241,060; 4,707,440; and U.S. patent applications Ser. Nos. 07/956,566 (filed on Oct. 5, 1992); 08/479,993 (filed on Jun. 7, 1995); and 08/574,443 (filed on Dec. 15, 1995), the contents of each of which is incorporated by reference in their entirety into this application. They can be introduced into the cell as viral particles or by a variety of physical means. These physical means include but are not limited to electroporation, calcium phosphate precipitation, DEAE-dextran, lipofection reagents, liposome reagents, polycations and ligand mediated uptake. The biological functions of said non-native nucleic acid segments can include but are not limited to marker genes, the expression of proteins and RNA coding for ribozymes, sense and antisense sequences. The donor cells of the chimeric organ can be normal or modified. The modifications can also include treatments of donor cells to alter their immunogenicity in a potential host or recipient. Those skilled in the art will readily appreciate that this can encompass so-called masking of epitopes.

In the case of the recipient's cells, these can be derived from a solid organ that is capable of regeneration. Among such regeneratable organs are liver or skin, although those skilled in the art may appreciate that others may exist with varying degrees of regenerating capabilities. It should be appreciated that the allogeneic or xenogeneic donor cells can be selected from such diverse members as liver, skin and pancreatic islets, or their combinations.

In one aspect of the present invention, a chimeric organ provides novel means of overcoming previous limitations imposed by species specificity of infectious agents. A host which is either resistant or otherwise incapable of supporting a normal infection by a pathogenic agent is transformed into a receptive host by the implantation of susceptible xenogeneic cells into the host. Because the species-specific pathogen infects and propagates within the xenogeneic implanted cells, the species limitation is removed, thereby eliminating the necessity of surrogate animal models which use related but non-identical pathogens.

In one aspect of the present invention, an animal model is used that would allow in vivo replication of HBV, hepatitis C or other human specific viruses that are trophic to the liver under conditions that resemble a normal infection in a human host. The method introduces normal human hepatocytes into a healthy small animal such as a mouse, rat or other useful animal that has normal liver function by means that include but are not limited to intrasplenic, intrahepatic, intraportal vein or intraceliac artery injection. The amount of hepatocyte implantation can be determined by the user by adjusting the dosage of xenogeneic hepatocytes and whether means are also employed that induce liver regeneration.

Thus, the present invention is extremely useful in providing a non-human animal model having or having incorporated therein any solid chimeric organ that is also provided by this invention. The animal model can be one that was previously incapable of supporting a viral or pathogenic infection of interest prior to having the solid chimeric organ incorporated into the animal, but which after the incorporation, the animal has been rendered capable of supporting such infection. The agent for such a viral infection comprises, for example, hepatotrophic viruses. Among such hepatotrophic viruses are any or all of the following viruses: hepatitis A viruses, hepatitis B viruses, hepatitis C viruses, hepatitis D viruses and hepatitis E viruses. Those skilled in the art will certainly appreciate that there will be or may be other hepatotrophic viruses which are also useful in the present invention. Among the agents for pathogenic infections are those which cause malarial infections, namely plasmodium which are preferred in the development of the animal model herein.

Implantation of the xenogeneic hepatocytes can be of a minimal nature that does not disturb the normal cycle of liver regeneration. In the absence of stimulation, intrasplenic injection of hepatocytes can create a chimeric liver with approximately 1% of the hepatocytes derived from an allogeneic or xenogeneic donor (Ledley et al. (1993), supra. When a higher representation of donor cells is desired the implantation can be performed in conjunction with methods that induce liver regeneration. These methods can include but are not limited to partial hepatectomy, portal vein ligature or adminstration of compounds that are hepatotoxic. Hepatotoxic compounds can include but are not limited to D-galactosamine, carbon tetrachloride and pyrrolizidine alkaloids. Some of these methods have been used previously to increase the efficiency of retroviral transformation (Kalecko, et al., Human Gene Therapy 2:27-32 (1991); Kay et al., Human Gene Therapy 3:641-647 (1992); and Bowling et al., Human Gene Therapy 7:2113-2121 (1996)) since integration requires dividing cells and a pyrrolizidine alkaloid has been used to increase the implantation frequency of syngeneic hepatocytes in a gene replacement study (Laconi et al., FASEB Journal 11(3):A104 (1997)). All of the foregoing are incorporated by their entirety into this application.

A novel non-invasive approach for increasing implantation efficiency would be the introduction of a nucleic acid containing a coding sequence for a hepatotoxin into recipient hepatocytes prior to the implantation process. Expression of the hepatotoxin induces replacement/regeneration of hepatocytes in the liver of the recipient. Hepatotoxic genes can include but are not limited to the uPA gene described previously and the Herpes thymidine kinase (TK) gene. Transient expression of uPA from an adenovirus vector has been used to increase the efficiency of retroviral transduction in hepatocytes (Lieber et al., Proc. Natl. Acad. Sci. (USA) 92:6210-6214 (1995)) and the Herpes TK gene has been introduced into tumor cells to render them sensitive to the actions of ganciclovir (Ezzedine et al., New Biol. 3:608 (1991)). Both publications are incorporated by reference herein. For nucleic acid sequences that directly provide for the synthesis of a toxic gene product (such as the uPA gene) implantation can proceed after allowance for sufficient gene expression time. For nucleic acid sequences that provide sensitivity to a conditionally toxic compound (such as the Herpes TK gene and ganciclovir), after a period allowed for expression, another period is used for administration and purging of the toxic compound.

In the present invention, the proportion of the native hepatocytes to be modified by the presence of a potential hepatotoxin can be determined by the user. For example, infection with adenovirus can be nearly 100% efficient with lower levels reached by using proportionally smaller viral inputs. On the other hand, the use of retroviruses is self-limiting with at best only 1% of hepatocytes being transduced in mice (Kay et al., Human Gene Therapy 3:641-647 (1992)) and only 5-15% being transduced in rats (Ferry et al., Proc. Natl. Acad. Sci. (USA) 88:8377-8381 (1991); Moscioni et al., Surgery 113:304-311 (1993); and Kolodka et al., Somatic Cell Mol. Genet. 19:491-497 (1993)). All of the foregoing are incorporated by reference herein. Expression of the hepatotoxic gene can be transient or it can be derived from a stable integration of a nucleic acid. Adenovirus infection is an example of the former and retroviral infection is an example of the latter. Examples of non-viral means of gene delivery include but are not limited to liposomes and ligand mediated transduction. After the hepatotoxic gene is introduced and expressed, the cells to be engrafted can be administered. Similar to the situation described by the transgenic uPA mice, the death of the transfected cells should allow a higher replacement rate and further growth of the implanted cells. Also, treatments with hepatotoxins and/or engraftments can be repeated to increase representation of implanted cells. Methods useful for repeated administrations of gene therapy reagents are described in U.S. patent application Ser. No. 08/808,629 (filed on Feb. 28, 1997), the contents of which are incorporated by reference. These enhanced engraftment rates may have utility when a higher amount of liver damage by a viral infection or the effects of the spread of a virus need to be observed. In addition, increased implantation efficiency can be achieved by using a combination of two or more of the methods described above.

A chimeric liver model system for HBV therapeutics will require the acceptance of human hepatocytes engrafting within the mouse liver. The lack of an immune response can be due to a genetic defect that results in an immune compromised host or it can be induced in an otherwise immunocompetent host. Genetically determined immunotolerant hosts can include but are not limited to SCID mice (Bosma et al., Ann. Rev. Immunol. 9:323-350 (1983)) and RAG-2^{-/-} mice (Shinkai et al., Cell 68:855-867 (1992)). Both articles are incorporated by reference in this disclosure. Although susceptible to a variety of infections, these animals are healthy when maintained in isolation.

Alternatively, immunotolerance can be an induced in the host animal. This can either be a general immunosuppression or a specific suppression of an immune reaction to particular epitopes. Methods for inducing general immunosupression can include but are not limited to drugs such as FK506, cyclosporin A and rapamycin to make the host amenable to acceptance of xenogeneic hepatocytes.

A number of systems can be used in the present invention to allow tolerance to a specific antigen or set of antigens while preserving a capability of immune response to other antigens. These can include but are not limited to neonatal tolerance, thymic tolerance, T cell depletion or inactivation and oral tolerization as well as combinations thereof. Means of carrying out these methods are described in U.S. patent application Ser. No. 08/808,629, supra. In a preferred mode, the animal is made receptive to the implantation of allogenic or xenogeneic hepatocytes by an oral tolerization program where there is introduction of allogeneic or xenogeneic hepatocytes into the intended recipient prior to implantation. Because of what has been termed the “bystander effect”, the presence of foreign antigens in general from the cells used for the tolerization should be sufficient to induce a tolerance of implanted allogeneic or xenogeneic hepatocytes that may have other antigenic determinants as well. If a more controlled tolerization program is desired, the same allogeneic or xenogeneic source used for the implantation can be us used as a source for the tolerization as well. Although intact hepatocytes are needed for the implantation, lysates of cells have been found to be as effective as whole cells for induction of oral tolerance, so once cells have been implanted, the remainder of the hepatocytes can be stored frozen and used as a continual source to maintain tolerization.

In systems that induce tolerization to epitopes that are specific to the implanted cells, challenge by unrelated antigenic determinants at a later time still elicits an immune response showing that the tolerance is specific to the implants and not a general downregulation of the immune system. This method should be particularly useful for deriving a model system where implanted human hepatocytes are tolerated in a xenogeneic host, but an immune response to HBV viral antigens is desired, thus bringing the model closer to a normal HBV infection in a human host.

On the other hand, transplant recipients that have either genetic or induced general immunosuppression, can serve as models for development of therapeutic regimens for HBV infected patients who have an immune deficiency condition such as AIDS. They can also serve in studies that are designed to examine the effects of viral therapeutic agents in the absence of either help or hinderance generated by an immune reaction to the viral infection.

In another aspect of the present invention, allogeneic or xenogeneic hepatocyte implantation into an immune compromised recipient is accompanied by transplantation of lymphocytes and or hematopoietic stem cells to provide an immune response. For example, infection of a chimeric liver by HBV can be carried out under conditions where donor lymphocytes are derived from patients with varying levels of response to an HBV infection i.e. cells from naiive donors, or from patients with HBV immunization, resolved HBV, chronic HBV infection or a fulminant HBV infection. The adoptive immune reaction can be against the HBV antigens and/or an HBV induced autoimmune response to hepatocytes. The autoimmune response can be against epitopes that are present only in the implanted donor cells or they can be epitopes that are shared by the native hepatocytes.

The present invention has utility for HBV and human hepatocytes in a mouse recipient, and in addition it provides a model for other diseases that may be specific to humans which lack appropriate small animal models. These disease processes can be specific to the liver or other organs that allow engraftment of foreign cells. As described previously, a model system for the exoerythrocyte phase of Plasmodium falciparum that involves residence and propagation within hepatocytes, was carried out by ectopic transplantation of human hepatocytes under the kidney capsule (Sacci et al. (1992), supra). The present invention overcomes limitations due to the use of an ectopic location by creating a chimeric liver that would provide a more normal environment for growth of malarial sporozoites.

In addition, the present invention is not confined to human pathogens and will enjoy utility with any infectious process that is confined to only a few specific species where it would be advantageous to use a different species, which is not infectable, as the model.

It is a further aim of the present invention to provide surrogate models for gene therapy that can be used for individual patients. Due to a lack of protocols for pre-testing of potential subjects, gene therapy trials currently involve testing subjects directly by in vivo or ex vivo introduction of genetic therapy reagents. In one aspect of the present invention, cells that are intended to the targets of a gene therapy protocol are obtained from a potential gene therapy candidate and transferred into a host to create a chimeric organ. A gene therapy protocol can then be administered to the cells of the gene therapy candidate either prior to or after implantation into a non-human host. The present invention allows the target cells in the chimeric organ to be tested for the extent and efficiency of the expression and or lack of adverse effects due to the gene therapy protocol while the target cells are maintained in a native environment in a surrogate non-human animal prior to the use of the human subject for gene therapy. This is an improvement over prior art since individual subjects may have different susceptibilities to gene therapy reagents; these parameters can include variations in the levels of uptake and expression as well as the stability of expression.

The solid chimeric organs of the present invention and described herein can be prepared from recipient organs which comprises recipient cells. Again, the solid chimeric organs comprise both recipient cells and implanted allogeneic or xenogeneic donor cells from a non-homologous organ or tissue. When so prepared, the recipient organ has been reduced in size or the total number of recipient cells in the recipient organ have been reduced. The reduction in the organ or the number of cells can be effected prior to or during implantation of the donor cells. The solid chimeric organ prepared from a recipient organ can further comprise implanted donor cells from a homologous organ or tissue. The recipient cells described hereinabove are typically derived from a solid organ that is itself capable of regeneration, e.g., liver or skin, although the person skilled in the art will appreciate that there may be other solid organs that are capable of regeneration. Furthermore, the allogeneic or xenogeneic donor cells described above can take various forms or be derived from various sources, including liver, skin and pancreatic islets, or combination of the foregoing. It is a further aim of the present invention to provide a system for storage or expansion of human cells. The in vivo growth of the implanted cells as part of a chimeric organ in a xenogeneic host allows cells to maintain their native capabilities without loss or gain of properties that are associated with in vitro growth or maintenance. For instance, although hepatocytes are continuously replenished in the liver, primary hepatocytes have only a very limited number of generations in vitro, despite the nutrients and co-factors that are added to the in vitro media. This disparity in viability indicates a lack of appropriate factors and/or structures that are necessary for maintaining the growth capacity of the hepatocyte cultures and the primary cultures undergo terminal differentiations that limit their lifespans.

In addition, in vitro growth allows or selects for cell transformation as indicated by an association of tumorgenicity with treatment of fibroblast cells with gene therapy reagents even after only a limited expansion in vitro (Chang et al., Mol Biol. Med. 7:461-470 (1990; and Seppen et al., Human Gene Therapy 8:27-36 (1997)). Both are incorporated by reference herein. The present invention avoids the potential for these deleterious effects by maintaining the growth of cells as part of a chimeric organ in an in vivo allogeneic or xenogeneic temporary host. In this way all of the normal metabolic and structural factors are present to maintain the implanted cells as they would in the native organ. After temporary in vivo storage as part of a chimeric organ, the donor cells can be reimplanted into the original subject or they can be transplanted into a new host. Prior to being reimplanted into the original host, the cells can be maintained unmodified or they can be subjects of gene therapy depending upon the intended use. Markers native to the donor cell or artificially added markers can be used as means of identification and selection of donor cell when separating them from the native cells of the chimeric organ.

In another aspect of the present invention, methods and compositions are described for a heterologous chimeric organ that is comprised of cells native to such an organ and cells that are not normally located in such an organ. In one aspect of the present invention, the integration of the ectopically implanted allogeneic or xenogeneic cells into the target organ is carried out after treatment of the target organ with means that remove or induce loss of part of the target organ. In another aspect, the ectopically implanted allogeneic or xenogeneic cells have been modified to contain one or more non-native nucleic acid segments or one or more native nucleic acid segments capable of non-native expression. These means can include all of the methods described previously for increasing representation of allogeneic or xenogeneic cells in a chimeric organ.

The present invention further provides other two other processes for preparing the solid chimeric organ described in this disclosure. In one important embodiment, the process comprises the steps of: i) removing cells from an organ or tissue of a donor; and ii) introducing into the removed donor cells one or more non-native nucleic acid segments or one or more native nucleic acid segments capable of non-native expression in a target cell. The non-native nucleic acid segments or the native nucleic acid segments will be capable of eliciting at least one biological effect non-native to said cell. In a third step iii) the donor cells into which the segment or segments have been introduced will be implanted into a desired organ of an allogeneic or xenogeneic host.

In another important aspect, the invention at hand provides a process for preparing a solid chimeric organ. This process comprises the steps of: i) removing cells from an organ or tissue of a donor; ii) implanting the removed donor cells into a desired organ of an allogeneic or xenogeneic host; and iii) introducing into the implanted donor cells one or more non-native nucleic acid segments or one or more native nucleic acid segments capable of non-native expression in a target cell. The non-native nucleic acid segment or the native nucleic acid segments, as the case may be, will be capable of eliciting at least one biological effect non-native to the cell.

In the case of either of the just-described processes, the introducing and implanting steps can be carried out at about the same time, or they can be carried out at different times. Introduction can be carried out by any conventional means known in the art, including viral-mediated gene delivery or non-viral mediated gene delivery. Viral-mediated gene delivery can be mediated by an unmodified virus or even a virus derivative. Such a virus or virus derivative can take any number of forms or be any member such as the group consisting of retroviruses, adenoviruses, Adeno-associated viruses (AAV), herpes simplex viruses, Epstein-Barr viruses (EBV), hepatotrophic viruses, polioviruses and vaccinia viruses, or a combination of any of the foregoing. As described supra, the hepatotrophic viruses comprise any of the following members or their combination: hepatitis A viruses, hepatitis B viruses, hepatitis C viruses, hepatitis D viruses and hepatitis E viruses.

Where the use of non-viral mediated delivery is contemplated in accordance with the present invention, any number of conventional means may be successfully applied. These means include but are not limited to calcium phosphate transfection, DEAE-dextran transfection, liposome-mediated transfection, lipofection, electroporation, ligand-mediated delivery, microinjection and PEG-mediated fusion, or combinations thereof.

Another important process provided by this invention is that of preparing a solid chimeric organ from a recipient organ. The steps of this process include or comprise (a) reducing the size of the recipient organ or reducing the total number of recipient cells in the recipient organ; (b) providing allogeneic or xenogeneic donor cells from a non-homologous organ or tissue; and (c) implanting those allogeneic or xenogeneic donor cells provided from previous step (b) into the recipient organ. The providing step (b) or the implanting step (c) or both, can further comprise donor cells from a homologous organ or tissue. Furthermore, the recipient organ can comprise a solid organ capable of regeneration, such as the liver or skin, or others that may be known or apparent to those skilled in the art. The allogeneic or xenogeneic donor cells can be diverse, and may be selected from liver, skin and pancreatic islets, or combinations thereof.

Organs such as liver exhibit homeostasis such that after loss of a portion of a liver there is induction of growth factors that encourage a repopulation by cells remaining in the organ until a liver is regenerated that is approximately of the same size as the original liver. Thus, the vacancy created by the loss of part of a liver exists both as a physical space and as a metabolic signal. As a result when non-native cells are introduced into an organ that has been subjected to such treatments, they increase in amount due to the availability of space and presence of growth factors.

In the present invention, the same factors that are advantageous for integration of non-native cells into a reduced organ are utilized to introduce and integrate heterologous cells into the organ. After one or more of the treatments described above for reduction of the size or amount of the recipient organ, heterologous cells either by themselves or in conjunction with cells that are native to such an organ are introduced into the recipient organ. Heterologous cells can be native to the subject but non-native to the organ. An example of this would be pancreatic islet cells taken from a subject and implanted into the liver of the same subject. Heterologous cells can also be non-native to the subject and non-native to the organ or equivalent thereof. An example of this would be a pancreatic islet cells taken from a subject (donor) and implanted into the liver of a different subject (recipient).

Growth of either native cells or accompanying implanted cells will allow the integration and potential growth of the ectopically implanted cells in the regenerating organ. This will provide both structure as part of an integrated organ and access to angiogenesis induced by the regeneration. Any solid organ that is capable of regeneration can serve as a target organ. Of especial utility will be any heterologous cells or tissues whose functionality is not primarily dependent upon its location such that access to the hematic or lymph systems of the recipient establishes substantial functionality. Examples of such cells or tissues include but are not limited to pancreatic islets and other glands.

When there is an immunological reaction to the heterologous cells comprising part of the heterologous organ, any of the methods described previously for general immunosuppression, specific immunosuppression, tolerization or any combination thereof can be employed. It is also understood that an organ can be both chimeric and heterologous when it is comprised of implanted cells that are not native to such an organ and allogeneic or xenogenic cells that are native to an equivalent of the said organ.

In another embodiment of the present invention, the chimeric organs are generated by organ reduction followed by implantation of cells that in themselves are of chimeric nature. Creation of such a chimeric cell can be accomplished by the fusion of cells with different gene expression patterns thereby creating chimeric cells that display phenotypic properties derived from each of the parental cells. The means of the fusion event can be carried out by a number of ways that have been described in the literature. These can include but are not limited to fusions mediated by PEG or inactivated Sendai virus. These and other methods are described more fully by Shaw, J. W. (ed.) (1982) in Techniques in Somatic Cell Genetics (Plenum Press, New York, N.Y.). The publication by Shaw is incorporated by reference herein. The cells fused together can be of the same type but from different subjects. An example of this could be a fusion of mouse hepatocytes and human hepatocytes. The cells fused together can be of different types but the same subject. An example of this could be a fusion of a human lymphocyte and a human pancreatic islet. The cells fused together can be of different types and different subjects. An example of this could be a mouse hepatocyte and human pancreatic islet. Different subjects can be members of the same species or they can be of different species. The chimeric cell can be used without selection or if there is instability of the makeup of the cell there can be selection for cells that exhibit one or more of the characteristics intrinsic to each member of the fusion. For example, a fusion of a hematopoeitic stem cell with a an islet cell could be selected by antibodies specific for each cell type.

Creation of a chimeric cell can also be accomplished by the introduction of one or more chromosomes, segments thereof, isolated genes and artificial constructs (Klobutcher and Ruddle, Ann. Rev. Biochem 50:533-554 (1981) and Goodfellow et al. in Genome analysis—A Practical Approach (Davies, K. E. Ed.), IRL Press, Oxford, pages 1-18 (1988)) where the recipient cell does not ordinarily exhibit a phenotype that is conferred by the introduced genetic material. The aforementioned publication and book are incorporated by reference herein.

Another important aspect of this invention concerns the non-tumorigenic immortalized human cell lines which have been modified by introducing one or more sequences derived from hepatotrophic viruses. Such hepatotrophic viruses have been described previously and can include hepatitis A viruses, hepatitis B viruses, hepatitis C viruses, hepatitis D viruses and hepatitis E viruses, or combinations of any of the foregoing. In another significant embodiment, the cell line provided by this invention can be capable of producing extracellular particles containing viral nucleic acid or the cell line can actually produce extracellular particles containing viral nucleic acid. Such extracellular particles can themselves be capable of producing cellular infection or produce cellular infection. In another aspect, these extracellular particles will contain one or more sequences not native to the hepatitis virus.

The present invention is also directed to the production of susceptible cells. These susceptible cells will be derived from non-susceptible cells. In one aspect, therefore, the susceptible cell will be derived from a non-susceptible cell that is a host in vivo for a virus or pathogen of interest. Such a non-susceptible cell will have been modified to render it susceptible to infection by the virus or pathogen of interest. The modification can comprise, for example, the introduction of one or more native or non-native nucleic acid segments into the cell. As described hereinabove, the native or non-native nucleic acid segment or segments can be produced by or they can be derived from a number of sources, including but not limited to one or more nucleic acid constructs, a viral vector, a mutated virus or wild type virus, modified or unmodified oligo- or polynucleotides and a chromosome or chromosomal segment, or combinations of any of the foregoing.

In a different aspect of the present invention, there is provided a non-human animal model having a susceptible cell derived from a non-susceptible cell that has been modified by implantation to render it susceptible to a viral or pathogenic infection. The non-susceptible cell comprises or is derived from a hepatocyte. In a preferred aspect, the hepatocyte is grown in vitro to render it non-susceptible.

Also provided herein is a susceptible cell derived from a non-susceptible cell that has been modified to render it susceptible to a viral or pathogenic infection. The modification can itself be effected or carried out by exposure to a cellular component which can include by way of example, whole cells, cellular membranes, extracellular matrix molecules, and soluble extracts or purified components therefrom, or a combinations of any of the foregoing. The soluble extracts or purified components can themselves include or comprise any of the following or combinations of any of the following: cytokines, lymphokines, hormones, immunological factors and growth factors.

The present invention also provides a susceptible cell derived from a non-susceptible animal, the non-susceptibility running to a virus or pathogen of interest. The cells of the non-susceptible animal have been modified in accordance with this invention to render them susceptible to an infection by the virus or pathogen of interest. The modification to these cells can comprise introducing one or more non-native or native nucleic acid segments into such cells, using techniques and protocols described supra. The identity of such nucleic acid segments are also as described above. Also uniquely provided by this invention is an animal model having one or more of such aforementioned susceptible cells. Such an animal model can be obtained through the direct introduction of the nucleic acid segments just described into potential host cells. The animal model can also be obtained by in vitro introduction of the nucleic acid segments described supra into potential host cells and then implanted thereafter into a suitable animal appropriate to serving as a model.

It is another aim of the present invention to develop cytopathic variants of HBV as markers for therapeutic regimes for HBV infection. Site specific mutagenesis of the p36 gene of a related virus, DHBV, has been used to create cytopathic mutants (Lenhoff and Summers (1994), supra). However these mutants are limited in their application since DHBV is unable to infect human cells. As targets for potential HBV therapeutic regimes, these mutants also suffer the limitations that DHBV differs substantially in gene organization and sequence from the human virus.

Thus, the present invention is significant for providing cytopathic mammalian viruses derived from a non-cytopathic virus. The derivation of such viruses can be carried out by any number of conventional means including those such as mutations, introduction of additional promoters, and incorporation of other viral nucleic acid sequences, or host cell nucleic acid sequences. The non-cytopathic virus from which the cytopathic mammalian viruses of the present invention are derived can comprise a hepatotrophic virus, which can itself comprise a hepadnovirus. A well-known example of a hepadnovirus that is preferred for this invention is the Hepatitis B Virus.

In a particular aspect of the present invention, mutations are introduced into the genome of HBV to allow the isolation and identification of cytopathic variants. Since they are derived from HBV, these cytopathic derivatives will be capable of infecting human cells while retaining a genomic sequence substantially identical to normal HBV. Although mutations at any given site in a hepadnavirus genome can have pleiotrophic effects due to overlapping coding sequences and multiple reading frames, sequence information derived from a large number of isolates of HBV have shown that there can be numerous point mutations, deletions and insertions that still allow HBV to be viable and capable of propagation. Naturally occurring mutants do not neccessarily represent the entire spectrum of potential HBV variants since cytopathic variants may be strongly selected against (Lenhoff and Summers (1994), supra).

In the present invention, HBV variants can be created by random or site specific mutagenesis. When using site specific mutagenesis, selected portions of the HBV can be targeted or sites throughout the genome can be tested for inducement of a cytopathic phenotype. The mutations can be point mutations, deletions or insertions. In a preferred mode, site specific modifications are made in the sequences coding for the pre-S1 gene, which codes for the HBV large envelope protein. Due to the redundancy inherent in the genetic code, the mutations can be chosen such that they create differences in the amino acid sequence of the pre-S1 gene while preserving the amino acid sequence of the gene products of the other reading frames. Mutants obtained by this method should have various phenotypes, some of which will be cytopathic.

Small or large scale screening of mutants can be performed by introduction of the mutated HBV genomic DNA into primary human hepatocytes or human hepatocyte cell lines. The means of introduction can include but are not limited to viral infections, electroporation, calcium phosphate precipitation, lipofection and liposomes. Characterization of the mutants can include assays for intracellular HBV DNA replication and HBV viral particle production as well as direct observation of cytopathic effects in the target cells. Methods for carrying out these assays are described by Lenhoff and Summers (1994), supra, and others. When the nature of the mutation allows the accumulation and release of progeny virus, subsequent infection by such viral particles can also serve as a means of introducing the mutant viral DNA into target cells.

When a propagative infection is desired, the most useful variants of this series will be cytopathic to the host cell and capable of releasing infectious viral particles. On the other hand, when a system is being used that does not involve multiple rounds of infection, mutants that are deficient in viral release but exhibit stronger cytopathic effects may have more utility.

A target cell is also provided in this invention. In this embodiment, the target cell has been rendered susceptible to a cytopathic event upon infection by a noncytopathic virus. The cell will have been modified by introducing at least one native or non-native nucleic acid construct thereinto. The native or non-native construct can be derived from a virus or a cell.

In the present invention, cytopathic HBV mutants which are replication defective can be used in the same manner described previously for other hepatotoxic means of organ reduction. This self-limiting loss of hepatic cells can then be used as part of a therapeutic regimen to increase representation of donor cells in the liver. The implanted donor cells can be unmodified healthy cells, or they can be modified by the introduction of one or more non-native nucleic acid segments, or one or more native nucleic acid segments capable of non-native expression.

In addition, the cytopathic HBV mutants of the present invention can be used in vitro or they can be used in vivo to test the efficacy of potential therapeutic agents. Therapeutic agents that can be tested with such mutants include but are not limited to ribozymes, antisense oligonucleotides, genes coding for ribozymes or antisense RNA, genes coding for therapeutic proteins and chemical compounds, all of which may contain various modifications known to those skilled in the art. These chemical compounds can include but are not limited to nucleoside analogs and other inhibitors of viral polymerases, protease inhibitors, and inhibitors of viral infectivity, assembly or release.

An example of an in vitro use would be to use a cytopathic HBV variant to test the potential resistance conferred on hepatocytes by HBV genetic anti-sense constructs, using successive infections or transfections as was performed previously with HIV-1 AS constructs (Liu et al., J. Virol. 71:4079-4085 (1997), incorporated by reference in this application). Because HIV is a cytopathic virus, the presence of effective AS resistance to HIV-1 provided a positive selection for a pool of cells that were completely resistant to HIV-1, and the same is predicted for HBV-resistant cell selection.

An example of an in vivo use would be to use a cytopathic HBV variant to infect a chimeric liver in a mouse where the human cells used to create the chimeric liver contain HBV genetic anti-sense constructs. After infection by cytopathic HBV, effective AS inhibition would allow the persistence of the human cells in the chimeric liver whereas AS constructs unable to inhibit the virus would allow the destruction and elimination of the human cells from the chimeric liver.

In addition to modifications of the HBV genome to create cytopathic variants, another aspect of the current invention is to achieve a virus dependent cytopathic event by modification of a target cell. A virus dependent cytopathic modification can be derived from the introduction of viral or host cell genes or gene segments into the target cell. The biological functions of the said viral or host genes or gene segments can be expressed transiently or they can be stable components of the target cells. The biological functions of the said viral or host genes or gene segments can comprise but are not limited to the following processes: nucleic acid replication, transcription, reverse transcription, and translation. The biological functions provided by the said viral or host genes or gene segments do not create a cytopathic event unless there is a further introduction of either virus or virally derived nucleic acids.

Examples of means that could be used to create a transient presence of said biological functions could be but are not limited to adenovirus vectors, DEAE-dextran, electroporation, calcium phosphate precipitation, lipofection, liposomes and ligand mediated uptake. Examples of means that could be used to create a stable presence of said biological functions could be but are not limited to Retrovirus and Adeno Associated Virus vectors, electroporation, calcium phosphate precipitation, lipofection, liposomes and ligand mediated uptake.

In a preferred mode of the present invention, the said genes or gene segments are derived from HBV and when these genes or gene segments are present in a modified cell, the introduction of HBV DNA that is not ordinarily cytopathic results in a cytopathic event in the modified cell. For example, alterations in either the properties of HBV gene products, the amount of HBV gene products, the ratios of certain gene products to each other or the ratio of certain gene products to one or more forms of HBV DNA in a cell could potentially create conditions that will lead to a cytopathic event in a cell.

In additon to modifications of a viral or host cell genome to create a virus dependent cytopathic event, another aspect of the present invention is to expose cells either in vivo or in vitro to reagents or agents that would initiate a cytopathic event in a cell that is undergoing viral replication or expression. Examples of these reagents and agents could include but are not limited to apoptosis factors and cells involved in immune surveillance. Examples of apoptosis factors could include but are not limited to tumor necrosis factor and fas ligand. Examples of immune surveillance cells could include but are not limited to CTLs that are specific for viral antigens. In the present invention, any of the means described to convert a noncytopathic viral infection into a cytopathic event can be used for testing of therapeutic agents, isolation of cells resistant to viral infection or organ depletion.

In another aspect of the present invention, immortalized cell lines that have lost the capability of being supportive for HBV infection are treated or modified such that they are competent for the uptake and expression of viral particles. The said modification or treatments can be carried out either in vivo or in vitro. Examples of in vitro treatment can include but are not limited to the introduction of a gene coding for an HBV receptor into the target cells, co-cultivation of non-susceptible target cells with primary hepatocytes or other liver derived cells and the growth and infection of non-susceptible target cells in the presence of components derived from livers. These aforesaid components can be either soluble extracts, cellular membranes, extracellular matrix molecules or combinations thereof. An example of an in vivo modification or treatment can include but is not limited to reimplantation of non-susceptible cells into a host animal to expose them to hepatic factors, support cells and architecture to restore susceptibility. In the present invention, the non-susceptible cells can be congeneic, syngeneic, allogeneic or xenogeneic with reference to the host animal.

The present invention is also important for providing a human cell infected or transfected by a noncytopathic virus. The human cell can be rendered susceptible to a cytopathic event by exposing it to a biologically active compound. Useful biologically active compounds include, for example, cytokines, lymphokines, extracellular matrices, hormones, apoptosis factors, and immunological factors, or a combination of any of the foregoing. A preferred cytokine comprises a tumor necrosis factor, and particularly preferred among the latter is TNFα. Among preferred apoptosis factors are soluble fas ligands.

In still yet another important aspect, the present invention provides a process of increasing implantation efficiency. Such efficiency can be achieved or effected by the following steps: providing a recipient subject having an organ treated to reduce its mass or volume; and implanting allogeneic or xenogeneic donor cells into such treated organ, the donor cell having been modified to contain one or more non-native nucleic acid segments, as described more fully hereinabove. The donor cells are typically taken from an organ of the donor corresponding to the treated organ in the recipient subject. The donor cells are also typically taken from an organ of said donor that is different from said treated organ in said recipient.

The examples that follow are given to illustrate various aspects of the present invention. Their inclusion by no means is intended to limit in any way the scope of this invention as more particularly defined by the claims.

### DESCRIPTION OF THE PREFERRED EMBODIMENTS

### EXAMPLE 1

### HBV Transfected Hepatocytes in a Chimeric Liver

Immortalization of primary hepatocytes

Primary hepatocytes were obtained from a healthy patient by normal perfusion methods and prepared as described by Ponder et al. (1991), supra. They were immortalized by transfection with DNA coding for SV40 T antigen and selected by continual passaging in Williams E media (GIBCO-BRL, Gaithersburg Md.) supplemented with 5% FBS (GIBCO-BRL, Gaithersburg, Md.), 2 mM L-Glutamine, 50 nM dexamethasone, 20 mU/ml insulin (Boehringer Mannheim, Indianopolis, Ind.), 100 units/ml Pennicilin, 100 ug/ml Streptomycin at 37° C. in 5% CO₂. One clone isolated by this method has been termed IHHA-1 and used for subsequent steps.

Assay for tumorigenic potential of IHHA-1

The IHHA-1 cell line was tested for the ability to form tumors in immunocompromised mice. RAG-2 -/-, a strain of homozygous SCID mice (Shinkai et al., Cell 68:855-867 (1992), incorporated by reference herein), were obtained from Taconic Farms (Germantown, N.Y.) and bred and maintained in isolation chambers. 2×10⁶ cells were injected subcutaneously into 5 different RAG-2 mice and the ability of the transformed cells to grow in the mice was assayed weekly by visual inspection for tumor growth. Up to 13 weeks after injection, no evidence was seen for the growth of tumors in 5 out of 5 mice indicating a low tumorigenic potential for the IHHA-1 cell line. In a control experiment when the same number of cells from hepatoma cell line HuH-7 (Nakabayaski, et al., Cancer Res. 42:3858-3863 (1982), also incorporated herein by reference thereto) were injected into 4 different RAG-2-/- mice, large visible tumors were generated in all 4 mice after only 4 weeks.

Procedure for selection of human cells that contain HBV DNA

A plasmid containing a complete copy of the HBV genome (adw serotype) was digested to completion with EcoR1 restriction enzyme to release unit length HBV DNA. The 3.2 kb linear HBV genome was gel-purified by electroelution followed by phenol extraction and ethanol precipitation. It was then re-ligated for 2 hours at 15° C. followed by 24 hours at 4° C. to produce circular HBV monomers and concatemers. 1×10⁵ IHHA-1 cells were plated onto collagen coated 66 mm dishes and at approximately 80% confluence (48 hours after plating) the cells were washed two times with serum-free RPMI medium (GIBCO-BRL, Gaithersburg Md.). Five ug of the HBV DNA was mixed with 1 ug of a co-transfection marker, pRc/CMVneo (Invitrogen, San Diego, Calif.), in 300 ul of serum free RPMI media. 20 ul of Lipofectamine (GIBCO)-BRL, Gaithersburg, Md.) was mixed with 300 ul of serum free RPMI media and then mixed with the diluted DNA and allowed to stand for 15 minutes at room temperature. The transfection mix was brought up to a final volume of 2 ml by the addition of serum free RPMI media and layered over the washed IHHA-1 cells. At five hours post transfection, the medium was removed and replaced with 2 ml of complete William's E medium. At twenty-four hours post-transfection, the the cells were trypsinized and resuspended at a concentration of 8×10³ cells/ml. One-half ml of the suspension was reseeded into each well of a 24 well plate. To select for transfectants, the medium was supplemented with 100 ug/ml G418 (GIBCO-BRL, Gaithersburg, Md.). The G418 concentration was increased to 300 ug/ml after 7 days.

Identification of HBV antigen producing clones

At 2 days and 28 days post-transfection, samples were removed from individual wells and tested for the presence of HBV surface antigen (HBsAg). The assays were performed at ENZO Clinical Laboratories (Farmingdale, N.Y.) by the same procedures that are used for clinical specimens. No significant levels of antigen production were seen with the samples taken two days post-transfection. The results for samples take at 28 days post-infection are shown in Table 1.

TABLE 1

| Pool # | S/N | HBsAg | Pool # | S/N | HBsAg |
|---|---|---|---|---|---|
| 247 | 2.76 | (+/−) | 259 | 2.15 | (+/−) |
| 248 | 1.92 | − | 260 | 1.50 | − |
| 249 | 1.92 | − | 261 | 1.68 | − |
| 250 | 1.72 | − | 262 | 1.77 | − |
| 251 | 1.72 | − | 263 | 1.57 | − |
| 252 | 1.50 | − | 264 | 1.98 | − |
| 253 | 1.88 | − | 265 | 2.36 | (+/−) |
| 254 | 1.58 | − | 266 | 1.81 | − |
| 255 | 1.81 | − | 267 | 2.17 | − |
| 256 | 1.46 | − | 268 | 1.56 | − |
| 257 | 1.33 | − | 269 | 9.69 | (+) |
| 258 | 1.48 | − | | | |

The highest reading (pool #269) was 9.69 versus a reading of 0.9 for a negative control taken 28 days post-transfection. Since this well most likely contained a pool of several G418 resistant clones, the cells from this well were trypsinized and re-plated in a 6 well culture dish and grown for two weeks in 300 ug/ml G418. Samples of cell media were tested for HBsAg as described earlier. The readings were 1.03, 1.00, 0.97, 0.99 0.98 and 50.21 respectively. The last well appeared to be enriched for HBsAg producing cells, and this culture was designated IHBV 6. Individual clones were selected from the IHBV 6 pool by seeding at an average density of one cell per 33 mm plate and testing the media for HBsAg as described previously. Results from this assay is given in Table 2.

TABLE 2

| Clone | S/N | HBsAg | Clone | S/N | HBsAg |
|---|---|---|---|---|---|
| 6.1 | 1.2 | − | 6.8 | 2.6 | (+/−) |
| 6.2 | 1.4 | − | 6.9 | 4.6 | (+) |
| 6.3 | 14.2 | (+) | 6.10 | 1.7 | − |
| 6.4 | 48.2 | (++) | 6.11 | 1.1 | − |
| 6.5 | 24.1 | (+) | 6.12 | 1.4 | − |
| 6.6 | 1.1 | − | 6.13 | 1.1 | − |
| 6.7 | 72.9 | (+++) | 6.14 | 1.1 | − |

Clone IHBV 6.7 gave the highest level of HBsAg production and was selected for all further work. The level of HBsAg produced by IHBV 6.7 is comparable to the levels achieved by HBV producing cell lines derived from hepatic tumors (Sells et al. (1987), supra; and Goodarz et al. J. Gen. Vir. 71:3021-3025 (1990), also incorporated herein). The high expression of HBsAg is a stable feature of IHBB 6.7 as demonstrated by a reading greater than 40.0 when the clone was retested after 12 more passages.

Characterization of IHBV 6.7

The production of viral particles with associated virion DNA and polymerase by the IHBV 6.7 clone was demonstrated by isolation of viral particles as described by Gerelsakhur et al., J Virology. 70:4269-4274 (1996)), and the incorporation of radioactively labelled nucleotides into endogenous HBV template DNA by the endogenous HBV polymerase was measured as described by Sells et al. (J. Vir. 62:2836-2844 (1988), incorporated by reference herein). As a control, Hep G2.2.15 cells which are known to produce complete HBV virions (Sells et al., 1988, supra) were processed and analyzed by the same procedure. The procedure and results are more fully described below.

Confluent plates of IHBV 6.7 or Hep G2.2.15 cells were washed with PBS and then the cells were lysed in 1 ml of 10 mM Tris, 1 mM EDTA, 0.25% Triton X-100, 50 mM NaCl, 8% sucrose by incubation for 10 minutes at 37° C. Lysates were scraped from the plates into centrifuge tubes and clarified by centrifugation at 12,000 rpm in an Eppendorf microfuge. Supernatants were transferred to new tubes and adjusted to 7 mM MgCl₂ in a volume of 800 ul. Fifty ug Dnase I (Boehringer Mannheim, Indianopolis, Ind.) and 20 ug Rnase A (Boehringer Mannheim, Indianopolis, Ind.) were added and incubated at 37° C. for 30 minutes to remove cellular DNA and RNA. HBV Nucleocapsids were precipitated by the addition of 330 ul of 26% PEG 8000 (Sigma, st. Louis, Mo.), 1.4 M NaCl, 25 mM EDTA and incubation on ice for 30 minutes followed by centrifugation at 12,000 rpm in an Eppendorf microfuge for 4 minutes. Pellets were resuspended in 200 ul 10 mM Tris, 1 mM EDTA, 50 mM NaCl per 100 mM plate starting material (˜5×10⁶ cells/plate).

45 ul of the purified nucleocapsid sample was mixed with 2 ul each of stocks of 10 uM dATP, dTTP and dGTP (Boehringer Mannheim, Indianopolis, Ind.), 2 ul of 1 M MgCl₂, 0.5 ul Triton X-100 and 2 ul (20 uCi) a³²P-dCTP (Amersham, Arlington Heights, Ill.). Incubation of the mixture was carried out at 37° C. for 2 hours. DNA was purified by two extractions with equal volumes of phenol, two extractions with equal volumes of 1:1 phenol/chloroform and ethanol precipitation. The entire contents of a reaction were loaded into the well of a 1.2% Agarose gel and subjected to electorophoresis in 1× Tris-Borate-EDTA buffer. The gel was dried for 2 hours and exposed to XAR film (Kodak, Rochester, N.Y.). Both the samples from the IHBV 6.7 and the HepG2.2.15 cells produced the same size pattern for the incorporated nucleotides, a smear of 3 kb and less. However, the signal that appeared for the sample from the IHBV 6.7 cells after only 24 hour autoradiography was stronger than the signal generated by the sample from the HepG2.2.15 cells after 7 days of exposure.

Since this assay is dependent upon both the presence of the HBV coded polymerase as well as an endogenous DNA template the positive result indicates that both are being synthesized by the IHBV 6.7 cell line and that they are present in intracellular viral nucleocapsids. In addition, the number of intracellular nucleocapsids containing both endogenous polymerase and endogenous HBV template is approximately 20 times higher in the IHBV 6.7 clone as compared to HepG2.2.15.

Introduction of HBV producer cells into RAG-2 mouse liver

For implantation, adult RAG-2 -/- mice (described above) were anaesthetized with intraperitoneal injection of sterile 2.5% 2-2-2 tribromoethanol solution (Aldrich Chemicals, Milwaukee, Wis.) in PBS at a dosage of 0.4 ml per 25 g of mouse body weight. Under sterile conditions in a laminar flow hood, a small (1 cm) incision was made in the left flank of each mouse; the exposed spleen was externalized by pulling out the underlying fat pad less than 1 cm. IHBV 6.7 cells (described above) were trypsinized, washed with PBS and resuspended in PBS at a concentration of 1×10⁷ cells/ml. For each subject, 1×10⁶ IHBV 6.7 cells (in 0.1 ml PBS) were injected into the spleen by a tuberculin syringe with a 27½ gauge needle. The inner muscle and outer skin layers were closed with 5.0 chromic gut cuticular sutures and the incision covered with sterile gauze. The survival rate for the implantation subjects was 100%.

Detection of HBV in chimeric mice

After the implantation of the IHBV 6.7 cells, blood samples were taken from the tail vein at various intervals and assayed for the presence of HBV DNA. For some experiments, DNA was extracted from whole blood while in other experiments the DNA was extracted from cell pellets. For analysis of whole blood, DNA was extracted by addition of 100 ul sample to 400 ul of lysis mixture (50 mM Tris [pH 8.0], 100 mM EDTA, 0.5% SDS and 100 ug/ml Proteinase K [Boehringer Mannheim, Indianopolis, Ind.]), followed by overnight incubation at 55° C. For cell pellets, whole blood was processed by centrifugation in an Eppendorf 5415C microfuge for 10 minutes at top speed and the pellet resuspended directly in 400 ul of the lysis mixture described above followed by overnight incubation at 55° C. The DNA was purified by phenol extraction, phenol/chloroform extraction and ethanol precipitation followed by resuspension in 30 ul 10 mMTris, 1 mMEDTA.

Samples were amplified using primers (HBO1 and HBO2) for conserved sequences in the core region (ENZO Diagnostics, Farmingdale, N.Y.). Amplification mixtures consisted of 5 ul sample, 5 ul of 10× Taq Buffer (Perkin-Elmer, Foster City, Calif.), 4 ul 25 mM MgCl₂, 0.25 ul each of 25 mM dATP, dTTP, dCTP and dGTP (Perkin-Elmer, Foster City, Calif.), 33.5 ul dH2O, 1 ul HBO1, 1 ul HBO2 and 0.25 ul Taq Gold (Perkin-Elmer, Foster City, Calif.). The thermocycle profile for PCR amplification was as follows: 94° C. for 8′, 95° C. for 2′, 55° C. for 1′, 74° C. for 2′, followed by 8 cycles of 95° C. for 55″, 58° C. for 50″, 74° C. for 2′, followed by 8 cycles of 95° C. for 55″, 65° C. for 50″, 74° C. for 2′ and one cycle of 95° C. for 55″, 58° C. for 50″, 74° C. for 8′. Evaluation of amplification was performed using the HBV Microplate Hybridization Assay kit from ENZO Diagnostics (Farmingdale, N.Y.) following the manufacturers instructions. The results of some of these experiments are shown in Table 3.

TABLE 3

| | Assay results of PCR amplification of | | | | |
|---|---|---|---|---|---|
| | blood samples after injection of | | | | |
| Mouse | IHBV 6.7 cells | | | | |
| (Rag II-/-) | 3 days | 4 days | 7 days | 10 days | Experiment # |
| 1 | 0.21 | | | | 1 |
| 2 | 0.12 | | | | 1 |
| 3 | 0 | | | | 1 |
| negative | .03 | | | | 1 |
| control | | | | | |
| 4 | | 0.24 | | 0.43 | 2 |
| 5 | | 0.18 | | 0.25 | 2 |
| 6 | | 0.30 | | 0.32 | 2 |
| 7 | | 0.16 | | 0.34 | 2 |
| 8 | | 0.30 | | 0.03 | 2 |
| negative | | 0 | | 0 | 2 |
| control | | | | | |
| 9 | | | 0.5 | | 3 |
| 10 | | | 0.5 | | 3 |
| 11 | | | 0 | | 3 |
| negative | | | 0 | | 3 |
| control | | | | | |

This example demonstrated that in one group of injected mice there was persistence of detectable amounts of HBV DNA in circulating blood in 4 out of 5 subject 10 days after the intrasplenic injection of IHBV 6.7 cells indicating the continued presence of the HBV producing cells and/or HBV viral particles at that timepoint.

### EXAMPLE 2

### In Vivo Infection of Human Hepatocytes with HBV in a Chimeric Liver

Implantation of human hepatocytes into RAG-2 mice

Healthy liver donors from U. Nebraska, negative for both HBV and HIV, were used as a source of primary hepatocytes. Cells were isolated by perfusion of liver tissue (Berry et al., J Cell Biol 43:506-520 (1969), also incorporated by reference in this application), washed in PBS and collected by centrifugation at 1,000 rpm in a clinical table top centrifuge. 7×10⁶ primary hepatocytes were resuspended in 0.6 ml of PBS and 0.2 ml of this suspension was injected intrasplenically into RAG-2 mice as described in Example 1.

In vivo infection of chimeric mice with HBV positive serum

Serum that had been identified as HBsAg positive was obtained from ENZO Clinical Laboratories (Farmingdale, N.Y.) and a pool was created from sera from 10 individual patients. The serum pool was injected peritoneally with a 27½ gauge needle into two chimeric mice four months after the engraftment described above. As a control, two RAG-2 mice that had not been engrafted were also injected with the HBV positive serum.

Detection of HBV DNA in infected mice

At intervals after injection of the HBV positive serum, blood is obtained from the tail of the HBV infected mice under sterile conditions. Analysis of the presence of HBV DNA sequences is carried out by the purification, PCR amplification and microplate assays for HBV DNA described previously in Example 1.

### EXAMPLE 3

### Implantation with HBV Infected Primary Hepatocytes

Infection of primary hepatocytes in vitro

Primary hepatocytes are obtained and processed as described in Example 2 except that they are grown in vitro for a short time to allow an ex vivo infection. Infectious serum is obtained as described in Example 2 and used to infect the primary hepatocytes after the viral particles have been treated with staph V8 protease as described by Lu et al, J. Virol. 70:2277-2285 (1996), also incorporated herein by reference). Twenty four hours after exposure of the primary hepatocytes to the modified HBV virions, the cells are washed with PBS, trypsinized and inoculated into RAG-2 -/- mice as described previously. Assessment of the presence of HBV proliferation in the chimeric subject is carried out by PCR amplification followed by microplate assays for HBV DNA in blood samples as described previously in Example 1.

### EXAMPLE 4

### Implanted Xenogeneic Hepatocytes as Model for Malarial Infection

Preparation and infection of mice with chimeric livers

Implantation of primary hepatocytes in RAG-2 mice is as described in Example 2. Infection with Plasmodium falciparum is as described in Sacci et al., (1992), supra. Successful infection of the human hepatocytesis tested by microscopic examination of liver tissue biopsies for merozoites and immunofluorescent detection of specific malarial antigens as described by Sacci et al. (1992), supra.

Many obvious variations will be suggested to those of ordinary skill in the art in light of the above detailed description and examples of the present invention. All such variations are fully embraced by the scope and spirit of the invention as defined more particularly by the claims that follow.

What is claimed is:

1. A solid chimeric organ comprising:
i) recipient cells; and
ii) donor cells comprising allogeneic or xenogeneic cells, said allogeneic or xenogeneic cells modified to contain one or more nucleic acid segments capable of exhibiting at least one biological property non-native to said donor cell.

2. The solid chimeric organ of claim 1, wherein said nucleic acid segments are native or non-native to said donor cells.

3. The solid chimeric organ of claim 1, wherein said nucleic acid segments are produced by or derived from a member selected from the group consisting of one or more nucleic acid constructs, a viral vector, a mutated virus or wild type virus, modified or unmodified oligo- or polynucleotides and a chromosome or chromosomal segment, or a combination of any of the foregoing.

4. The solid chimeric organ of claim 1, wherein said chromosome or chromosomal segment is from a cell taken from a different donor or taken from a cell from a different organ in said recipient.

5. The solid chimeric organ of claim 1, wherein said donor cells are chimeric.

6. The solid chimeric organ of claim 5, wherein said chimeric donor cells are derived from a first and second parental cells which are phenotypically different from each other, and said chimeric donor cells exhibit at least two phenotypic characteristics, at least one of which characteristics is derived from said first parental cell and at least one other characteristic is derived from said second parental cell, and wherein said first or said second parental cells are allogeneic or xenogeneic with respect to said recipient cells.

7. The solid chimeric organ of claim 1, wherein said nucleic acid segment or segments have been introduced into said donor allogeneic or xenogeneic cells prior to the formation of said chimeric organ.

8. The solid chimeric organ of claim 1, wherein said nucleic acid segment or segments have been introduced into said donor allogeneic or xenogeneic cells after the formation of said chimeric organ.

9. The solid chimeric organ of claim 1, wherein the non-native nucleic acid segment has been introduced into said donor allogeneic or xenogeneic cells during the formation of said chimeric organ.

10. The solid chimeric organ of claim 1, wherein said recipient cells are derived from a solid organ capable of regeneration.

11. The solid chimeric organ of claim 10, wherein said solid organ comprises liver or skin.

12. The solid chimeric organ of claim 1, wherein said allogeneic or xenogeneic donor cells are selected from the group consisting of liver, skin and pancreatic islets, or combinations thereof.

13. The solid chimeric organ of claim 1, wherein said biological property is selected from the group consisting of DNA synthesis, DNA replication, promoter function, DNA transcription, DNA translation, reverse transcription, ligation, post-transcriptional modification, transcriptional processing, post-translational processing, protein export, or combinations thereof.

14. A non-human animal model having the solid chimeric organ of claim 1.

15. The non-human animal model of claim 14, wherein said animal was incapable of supporting a viral or pathogenic infection of interest prior to having the solid chimeric organ of claim 1, and wherein said animal is rendered capable of supporting said viral infection after having said chimeric organ.

16. The non-human animal model of claim 15, wherein the agent for said viral infection comprises hepatotrophic viruses.

17. The non-human animal model of claim 16, wherein said hepatotrophic viruses are selected from the group consisting of hepatitis A viruses, hepatitis B viruses, hepatitis C viruses, hepatitis D viruses, hepatitis E viruses, or a combination of any of the foregoing.

18. The non-human animal model of claim 15, wherein the agent for said pathogenic infection comprises plasmodium.

19. A solid chimeric organ prepared from a recipient organ, said solid chimeric organ comprising:
(i) recipient cells, and
(ii) implanted allogeneic or xenogeneic donor cells from a non-homologous organ or tissue,
wherein said recipient organ has been reduced in size or the total number of recipient cells in said recipient organ have been reduced prior to or during implantation of said donor cells.

20. The solid chimeric organ of claim 19, further comprising (iii) implanted donor cells from an homologous organ or tissue.

21. The solid chimeric organ of claims 19 or 20, wherein said recipient cells (i) are derived from a solid organ capable of regeneration.

22. The solid chimeric organ of claim 21, wherein said solid organ capable of regeneration comprises liver or skin.

23. The solid chimeric organ of claims 19 or 20, wherein said allogeneic or xenogeneic donor cells (ii) are selected from the group consisting of liver, skin and pancreatic islets, or combinations thereof.

24. A process for preparing a solid chimeric organ comprising the steps of:
i) removing cells from an organ or tissue of a donor;
ii) introducing into said removed donor cells one or more non-native nucleic acid segments or one or more native nucleic acid segments capable of non-native expression in a target cell, said non-native nucleic acid segments or said native nucleic acid segments being capable of eliciting at least one biological effect non-native to said cell; and
iii) implanting the donor cells into which said segment or segments have been introduced into a desired organ of an allogeneic or xenogeneic host.

25. A process for preparing a solid chimeric organ comprising the steps of:
i) removing cells from an organ or tissue of a donor;
ii) implanting said removed donor cells into a desired organ of an allogeneic or xenogeneic host; and
iii) introducing into said implanted donor cells one or more non-native nucleic acid segments or one or more native nucleic acid segments capable of non-native expression in a target cell, said non-native nucleic acid segment or said native nucleic acid segments being capable of eliciting at least one biological effect non-native to said cell.

26. The process according to claims 24 or 25, wherein said introducing and implanting steps are carried out at about the same time.

27. The process according to claim 24 or 25, wherein said introducing step is carried out by a means comprising viral-mediated gene delivery or non-viral mediated gene delivery or both.

28. The process according to claims 27, wherein said viral-mediated gene delivery is mediated by an unmodified virus or a virus derivative.

29. The process according to claim 28, wherein said virus or virus derivative is selected from the group consisting of retroviruses, adenoviruses, Adeno-associated viruses (AAV), herpes simplex viruses, Epstein-Barr viruses, hepatotrophic viruses, polioviruses and vaccinia viruses, or a combination of any of the foregoing.

30. The process according to claim 29, wherein said hepatotrophic viruses are selected from the group consisting of hepatitis A viruses, hepatitis B viruses, hepatitis C viruses, hepatitis D viruses, hepatitis E viruses, or a combination of any of the foregoing.

31. The process according to claim 27, wherein said non-viral mediated gene delivery comprises a means selected from the group consisting of calcium phosphate transfection, DEAE-dextran transfection, liposome-mediated transfection, lipofection, electroporation, ligand-mediated delivery, microinjection and PEG-mediated fusion, or combinations thereof.

32. A process for preparing a solid chimeric organ from a recipient organ which comprises recipient cells, said process comprising the steps of:
(a) reducing the size of said recipient organ or reducing the total number of recipient cells in said recipient organ;
(b) providing allogeneic or xenogeneic donor cells from a non-homologous organ or tissue; and
(c) implanting said allogeneic or xenogeneic donor cells provided from step (b) into said recipient organ.

33. The process of claim 32, wherein said providing step (b) or said implanting step (c) or both, further comprises donor cells from a homologous organ or tissue.

34. The process of claims 32 or 33, wherein said recipient organ comprises a solid organ capable of regeneration.

35. The process of claim 34, wherein said solid organ capable of regeneration comprises liver or skin.

36. The process of claims 32 or 33, wherein said allogeneic or xenogeneic donor cells are selected from the group consisting of liver, skin and pancreatic islets, or combinations thereof.

37. A non-tumorigenic immortalized human cell line which has been modified by the introduction of a sequence derived from a hepatotrophic virus.

38. The non-tumorigenic immortalized human cell line of claim 37, wherein said hepatotrophic viruses are selected from the group consisting of hepatitis A viruses, hepatitis B viruses, hepatitis C viruses, hepatitis D viruses, hepatitis E viruses, or a combination of any of the foregoing.

39. The non-tumorigenic immortalized human cell line of claim 37, said cell line is capable of producing extracellular particles containing viral nucleic acid or produces extracellular particles containing viral nucleic acid.

40. The non-tumorigenic immortalized human cell line of claim 39, wherein said extracellular particles are capable of producing cellular infection or produce cellular infection.

41. The non-tumorigenic immortalized human cell line of claim of 39, wherein said extracellular particles contains one or more sequences not native to hepatitis virus.

42. A susceptible cell derived from a non-susceptible cell that is a host in vivo for a virus or pathogen of interest, said non-susceptible cell having been modified to render it susceptible to infection by said virus or pathogen of interest, said modification comprising the introduction of one or more native or non-native nucleic acid segments into said cell.

43. The susceptible cell of claim 42, wherein said native or non-native nucleic acid segment or segments are produced by or derived from a member selected from the group consisting of one or more nucleic acid constructs, a viral vector, a mutated virus or wild type virus, modified or unmodified oligo- or polynucleotides and a chromosome or chromosomal segment, or a combination of any of the foregoing.

44. The animal model of claim 43, wherein said non-susceptible cell comprises or is derived from a hepatocyte.

45. The animal model of claim 44, wherein said hepatocyte was grown in vitro to render it non-susceptible.

46. A non-human animal model having a susceptible cell derived from a non-susceptible cell that has been modified to render it susceptible to a viral or pathogenic infection, said modification having been rendered by implantation.

47. A susceptible cell derived from a non-susceptible cell that has been modified to render it susceptible to a viral or pathogenic infection, said modification having been rendered by exposure to a cellular component selected from the group consisting of whole cells, cellular membranes, extracellular matrix molecules, and soluble extracts or purified components therefrom, or a combination of any of the foregoing.

48. The susceptible cell of claim 47, wherein said soluble extracts or purified components are selected from the group consisting of cytokines, lymphokines, hormones, immunological factors and growth factors, or a combination of any of the foregoing.

49. A susceptible cell derived from an animal having cells, said animal being non-susceptible to a virus or pathogen of interest, the cells of said animal having been modified to render such cells susceptible to an infection by said virus or pathogen, said modification comprising the introduction of one or more non-native or native nucleic acid segments into said cells.

50. An animal model having one or more susceptible cells of claim 49.

51. A cytopathic mammalian virus, said virus being derived from a non-cytopathic virus, said derivation comprising a means selected from the group consisting of a mutation, introduction of additional promoters, and incorporation of other viral nucleic acid sequences, or host cell nucleic acid sequences.

52. The cytopathic mammalian virus of claim 51, wherein said non-cytopathic virus comprises a hepatotrophic virus.

53. The cytopathic mammalian virus of claim 52, wherein said heptotrophic virus comprises a hepadnovirus.

54. The cytopathic mammalian virus of claim 53, wherein said hepadnovirus comprises Hepatitis B Virus.

55. A target cell which has been rendered susceptible to a cytopathic event upon infection by a noncytopathic virus, said cell having been modified by the introduction of at least one native or non-native nucleic acid construct.

56. The target cell of claim 55, wherein said native or non-native construct is derived from a virus or a cell.

57. A human cell infected or transfected by a noncytopathic virus, wherein said human cell is rendered susceptible to a cytopathic event by exposing to a biologically active compound selected from the group consisting of a cytokine, a lymphokine, extracellular matrix, a hormone, an apoptosis factor, an immunological factor, or a combination of any of the foregoing.

58. The human cell of claim 57, wherein said cytokine comprises a tumor necrosis factor.

59. The human cell of claim 58, wherein said tumor necrosis factor comprises TNFα.

60. The human cell of claim 57, wherein said apoptosis factor comprises soluble fas ligand.

61. A process of increasing implantation efficiency comprising the steps of:
providing a recipient subject having an organ treated to reduce its mass or volume; and
implanting allogeneic or xenogeneic donor cells into said treated organ, said donor cell having been modified to contain one or more non-native nucleic acid segments.

62. The process of claim 61, wherein said donor cells are taken from an organ of said donor corresponding to said treated organ in said recipient subject.

63. The process of claim 61, wherein said donor cells are taken from an organ of said donor that is different from said treated organ in said recipient.
